(19) United States
(12) Patent Application Publication
(10) Pub. No.: US 2018/0127700 A1
(43) Pub. Date: May 10, 2018

(54) Cell Culture Substrate for Rapid Release and Re-Plating

(71) Applicant: UNIVERSITY OF SOUTH FLORIDA, TAMPA, FL (US)

(72) Inventors: OLUKEMI O. AKINTEWE, GIBSONTON, FL (US); MICHAEL C. CROSS, TAMPA, FL (US); SAMUEL JAMES DUPONT, TAMPA, FL (US); NATHAN D. GALLANT, TAMPA, FL (US); RYAN G. TOOMEY, TAMPA, FL (US)

(21) Appl. No.: 15/576,102

(22) PCT Filed: May 23, 2016
(86) PCT No.: PCT/US16/33769

§ 371 Date: Nov. 21, 2017

Related U.S. Application Data
(60) Provisional application No. 62/164,963, filed on May 21, 2015.

(51) Int. Cl. C12M 1/00; C12M 3/00; C12M 1/12; C12N 5/073

(52) U.S. Cl. CPC C12M 23/20; C12M 21/08; C12N 2513/00; C12N 5/0603; C12M 25/14

(57) ABSTRACT

The invention pertains to devices and methods for rapid release of a patterned tissue module. The invention pertains to a device comprising a substrate and a pattern of shape-memory polymer fabricated upon the substrate, wherein the shape-memory polymer is converted to a deformed state by exposure to an external stimulus, for example, a change in temperature, pH, ionic strength, solvent, salt, surfactant, or an electric or magnetic field. The invention also pertains to a method of releasing a patterned tissue module, the method comprising the steps of plating cells in the device of the invention, culturing the cells to produce the patterned tissue module on the pattern of shape-memory polymer, applying the external stimulus to the device, and collecting the patterned tissue module released from the pattern of shape-memory polymer.

### CROSS-REFERENCE TO RELATED APPLICATION

The present application claims the benefit of U.S. Provisional Application Ser. No. 62/164,963, filed May 21, 2015, which is hereby incorporated by reference in its entirety, including any figures, tables, or drawings.

This invention was made with government support under Grant Numbers DMR-0645574, DMR-1056475, and DGE-0638709 awarded by the National Science Foundation. The government has certain rights in the invention.

### FIELD OF THE INVENTION

The present invention is directed to devices and methods for rapid release of patterned tissue modules from tissue culture substrates.

### BACKGROUND OF INVENTION

The invention relates to modular tissue engineering, a technique that utilizes tissue building blocks as modular units to construct biological tissues with specific architectural features. As an example, modular tissues can be created using cell sheets and assembled through stacking of layers to enhance formation of complex microstructural functional units such as microvascular networks, thereby augmenting integration and facilitating recovery. This approach aims to develop biomimetic engineered tissues that effectively recapitulates native tissues.

Two basic systems are available for growing cells in culture, namely, growing cells as monolayers on an artificial substrate or an adherent culture, or free-floating cells in the culture medium or a suspension culture. The majority of the cells derived from vertebrates must be cultured on a suitable substrate specifically treated to allow cell adhesion and spreading.

Cell culture involves dispersal of cells in the artificial environment where nutrient solutions and appropriate conditions of temperature, humidity, and gaseous atmosphere promote, the growth of cells on a suitable surface.

Harvesting cells from a culture conventionally occurs through scraping, enzymatic degradation of cell surface proteins, and thermo-responsive polymer, as described by Okano et al. (U.S. Patent Application Publication No. 2011/0207220) and Branner et al. (U.S. Pat. No. 5,693,520).

Scraping inflicts significant damage to cells. Enzymatic degradation moderately damages cells and a majority of cells recover. Release from a thermo-responsive polymer has not been shown to cause damage to cells. Cells are often exposed to hypothermic conditions for a significant duration of time, where the release time is typically in excess of 20 minutes and is up to about 90 minutes. Although the capability of thermo-responsive polymers to release cultured cells has been shown, short release time and/or release at temperatures near that optimal for mammalian cells has not been demonstrated.

### BRIEF SUMMARY

The invention provides advantageous devices and methods for rapid release of patterned tissue modules. In accordance with the subject invention, tissue modules can be fabricated and harvested via a strain-mediated process.

In one embodiment, the device of the subject invention comprises a substrate and a pattern of shape-memory polymer fabricated upon the substrate, wherein the shape-memory polymer is converted to a deformed state by exposure to an external stimulus, for example, a change in temperature, pH, ionic strength, solvent, salt, surfactant, or an electric or magnetic field.

The invention also provides a method of releasing a patterned tissue module. In one embodiment, the method comprises the steps of plating cells in the device of the invention, culturing the cells to produce the patterned tissue module on the pattern of shape-memory polymer, applying the external stimulus to the device, and collecting the patterned tissue module released from the pattern of shape-memory polymer.

In certain embodiments, the step of applying the stimulus is performed for about 5 seconds to about 30 seconds.

### BRIEF DESCRIPTION OF DRAWINGS

FIG. 1 is a schematic view of one embodiment of cell culture substrate for rapid release according to the invention.

FIGS. 2A-2C provide an example of shape-memory pNIPAAm polymer microbeams for tissue module culture and harvest. (A) Schematic of microbeam fabrication steps. (1) A PDMS template is placed on a methacrylated coverslip and (2) pre-polymer solution is flowed into the recesses. (3) The polymer networks are polymerized with 350 nm UV light before the PDMS template is removed. (B) Three-dimensional reconstructions from z-stacks of images taken by confocal microscopy of a surface-confined microbeam of low aspect ratio (AR=0.5) with a collapsed (37° C.) height of 25 μm and width of 50 μm (left), which transforms into a bulbous geometry (right) upon thermally initiated shape change at 27° C. Note microbeams with AR<1.0 were used. (C) Schematic of formation and release of tissue modules from shape-memory polymer microbeams. (1) Cells adhered to and conformed to the shape of the microbeams, organizing into geometric tissue modules. (2) Tissue modules released from microbeams upon expansion beyond a critical lateral strain.

FIGS. 3A-3B provide phase contrast images of tissue modules before (left) and after (right) microbeam expansion. (A) High cell density (ε=0.33) and (B) low cell density (ε=0.31) were compared for similar strains. Scale bars=100 μm.

FIG. 4 shows that surface strain regulates tissue detachment from pNIPAAm microbeams. Untreated, sodium azide (NaN₃), Y-27632, or DTSSP treated tissue modules were subjected to a range of lateral strain on shape-memory polymer microbeams. Each data point represents one experiment. The vertical dashed line indicates the 25% strain threshold for releasing untreated tissue modules.

FIGS. 5A-5D provide phase contrast images of tissue modules before (left) and after (right) microbeam expansion (about 3 min). (A) Untreated, ε=0.35; (B) sodium azide (NaN₃), ε=0.50; (C) ROCK inhibitor (Y27632), ε=0.59 and (D) integrin crosslinked (DTSSP), ε=0.71 treatments are shown. Arrows indicate detached tissue stripes. Scale bars=100 μm.

FIG. 6A-6B show a LIVE/DEAD viability assay indicating that the majority (about 94%) of the harvested and reattached cells remained viable after release from the shape-memory polymer microbeams.

### DETAILED DISCLOSURE

In the following detailed description, reference is made to the accompanying drawings, depicting exemplary, non-limiting and non-exhaustive embodiments of the invention. These embodiments are described in sufficient detail to enable those having skill in the art to practice the invention, and it is understood that other embodiments may be used, and other changes may be made, without departing from the spirit or scope of the invention. The following detailed description is, therefore, not to be taken in a limiting sense, and the scope of the invention is defined only by the appended claims. Like numbers refer to similar features of like elements throughout.

The invention relates to modular tissue engineering, a technique that utilizes tissue building blocks as modular units to construct biological tissues with specific architectural features. As an example, modular tissues can be created using cell sheets and assembled through stacking of layers to enhance formation of complex microstructural functional units such as microvascular networks, thereby augmenting integration and facilitating recovery. This approach aims to develop biomimetic engineered tissues that effectively recapitulates native tissues.

Responsive materials are used for generating tissue modules because of the ability to conveniently manipulate cell-surface interactions on culture supports. The cell-surface interactions may be manipulated by, for example, changes in environmental factors such as temperature, pH, ionic strength, solvent, salt, surfactant, or an electric or magnetic field. For example, grafted films of poly(N-isopropylacrylamide) (pNIPAAm), a thermally responsive polymer, can be used to produce cell sheets because this polymer undergoes a sharp volume-phase transition due to thermally mediated changes in the hydrophilic and hydrophobic interactions around its lower critical solution temperature (LCST) of 32° C. In comparison to conventional enzymatic release of cells with trypsin and ethylene diamine tetra-acetic acid (EDTA), the hydration of grafted pNIPAAm provides a slow, but non-destructive, approach for tissue harvest so that intact monolayers of cells can be formed.

In one embodiment, the invention provides devices and methods comprising a substrate and a pattern of a shape-memory polymer fabricated on the substrate for the production and rapid release of patterned tissue modules. Accordingly, one embodiment of the invention provides a device comprising a substrate upon which a pattern of shape-memory polymer is fabricated.

For the purpose of this invention a shape-memory polymer is a polymer that has the ability to return from a deformed state (temporary shape) to their original (permanent) state, wherein the conversion to the deformed state is induced by an external stimulus (trigger), such as a change in temperature, pH, ionic strength, solvent, salt, surfactant, electric or magnetic field.

Rapid production, versatility and scalability are important factors for in vitro construction of tissues and organs. The invention provides a shape-memory polymer cell culture device that demonstrates these properties. This device is based on patterned arrays of microscale protrusions (or microbeams) of cross-linked shape-memory polymer, for example, a polymer responsive to changes in temperature, pH, ionic strength, solvent, salt, surfactant, or an electric or magnetic field.

A purely mechanical, strain-based mechanism of detaching intact tissue modules from patterned arrays of shape-memory polymer structures is provided. The fabricated polymer structures release cells organized into geometric tissue modules via large lateral strains utilizing the surface-confined polymers' anisotropic swelling properties. This mechanically induced method of rapid release is advantageous in comparison to thermally responsive films and coatings. This mechanism can be extended to a variety of shape-memory materials, which allows for controlled and rapid release initiated by stimuli other than temperature, thus providing enhanced flexibility in the design of tissue engineering platforms. Thus, the invention provides a rapid method for recovery of tissue modules in an efficient manner, which can be applied to the modular construction of tissues for organ models and regenerative therapies.

Rapid tissue module release occurs on shape-memory surfaces via mechanical mechanisms that are unique to patterned shape-memory polymer cell culture supports. The invention can be used to form the diverse building blocks required for building robust multilayered tissues that are complex in architecture and can be used, for example, to enhance vascularization in thicker tissue grafts for organ repair or replacement.

3-D pNIPAAm microbeams having various swelling ratios were fabricated to investigate the effect of swelling-induced strain on tissue module release. The effect of cell density on cell detachment was also examined and to understand the mechanism of tissue release from these shape changing surfaces, the roles of metabolic activity and cytoskeletal contractility was investigated by probing the adhesive interface.

Further, increased viability of cells within the released tissue modules is provided. Thus, the subject invention facilitates fabricating and harvesting living tissue building blocks with intact organization and cell-cell connections that can be used to build complex 3-D tissues via the assembly of diverse tissue modules.

The shape-memory polymer can be fabricated into various patterns, for example, beams, discs, various regular geometric shapes (for example, circle, ellipse, triangle, square), irregular or random shapes, for example, shape of a skin burn of a patient. Additional patterns that can be useful according to the invention can be designed by a person of ordinary skill in the art based on specific purposes and such embodiments are within the purview of the claimed invention.

In one embodiment, the shape-memory polymer is a thermo-responsive polymer, for example, pNIPAAm. Conversion of pNIPAAm to a deformed state, for example, lateral swelling of the microbeams, occurs upon a change in temperature resulting in the expansion and distortion of the surface of shape-memory polymer.

In another embodiment, the shape-memory polymer is responsive to changes in pH, ionic strength, solvent, salt, surfactant, or an electric or magnetic field. In such embodiments, conversion of shape-memory polymer to a deformed state, for example, lateral swelling of the microbeams, occurs upon changes in pH, ionic strength, solvent, salt, surfactant, or an electric or magnetic field resulting in the expansion and distortion of the surface of shape-memory polymer.

Non-limiting examples of shape-memory polymers responsive to changes in temperature, pH, ionic strength, solvent, salt, surfactant, or an electric or magnetic field are provided by Hu et al. (2012) and Meng (2010), the disclosures of which are herein incorporated by reference in their entirety. Additional examples of shape-memory polymer responsive to changes in temperature, pH, ionic strength, solvent, salt, surfactant, electric or magnetic field are well known to a person of ordinary skill in the art and such embodiments are within the purview of the claimed invention.

The substrate upon which the pattern of shape-memory polymer is fabricated can be selected based on the stimulus to which the shape-memory polymer responds. For example, a pattern of a shape-memory polymer sensitive to a light stimulus can be fabricated on a glass substrate; a pattern of a shape-memory polymer sensitive to an electric field can be fabricated on a metal or semi-conductor substrate, and a pattern of a shape-memory polymer sensitive to a magnetic field can be fabricated on a metal substrate.

Non-limiting examples of substrates useful for preparation of the devices of the invention include, glass, silicone, polystyrene, polycarbonate, and metal. In certain embodiments, the surface of a substrate and/or shape-memory polymer pattern is treated with an appropriate material, for example, fibronectin or collagen, to facilitate attachment of cultured cells onto the substrate and/or shape-memory polymer. Additional substrates suitable for fabricating the devices of the invention are well known to a person of ordinary skill in the art and such embodiments are within the purview of the invention.

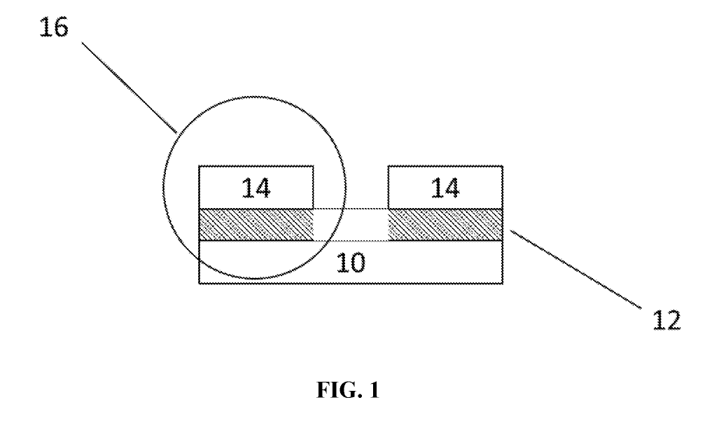

An example of the device for releasing a patterned tissue module is shown in FIG. 1. The device comprises a substrate (10) and a pattern of a shape-memory polymer (12). Patterns of shape-memory polymer can be used as a platform for generating tissue modules capable of being manipulated for cell-culture interactions on culture substrates. In one embodiment, the shape-memory polymer is thermally responsive polymer, for example, pNIPAAm.

The cell culture surface (10) can be subjected to treatment (14) by crosslinking or immobilization of functional groups or biomolecules that facilitate the binding of cells to the shape-memory polymer surface. In further embodiments, the chemical treatment may result in a relief pattern (16), the relief pattern comprising, for example, protruding features at the surface of the shape-memory polymer. The presence of a relief pattern, in some embodiments, can improve adhesion of cells onto the shape-memory polymer pattern.

A further embodiment of the invention provides a method of rapid release of patterned tissue modules from tissue culture substrates. The release mechanism described herein is mechanical in nature, caused by the deformation of the shape-memory polymer substrate (12) upon appropriate stimulation. For example, appropriately applied environmental stimuli may induce swelling of the pattern of the shape-memory polymer (14/16), subjecting the pattern to a mechanical strain. The swelling-induced deformation can in turn assert mechanical strain upon the cells attached to the surface (10), resulting in the release of cells. In some embodiments, the cell release from the stimuli-responsive, chemically modified culture surface may take 5 to 30 seconds, allowing rapid cell passaging.

Accordingly, in certain embodiments, the method of rapid release of patterned tissue modules comprises the steps of:

a) plating cells in a device comprising a substrate upon which a pattern of shape-memory polymer is fabricated, wherein the shape-memory polymer is converted to a deformed state by exposure to an external stimulus,

b) culturing the cells under appropriate conditions to produce a patterned tissue module on the pattern of shape-memory polymer,

c) applying the external stimulus to the device, and

d) collecting the patterned tissue module released from the pattern of shape-memory polymer.

Various methods of plating the cells are well known a person of ordinary skill in the art. For example, primary, secondary, or continuous cell cultures can be plated.

Conditions appropriate for the growth of the cells depend on the type of cells cultured and the intended use of the cultured cells. The cells can be cultured at an appropriate temperature, for example, 37° C., for an appropriate period of time, for example, 2-3 days to several weeks. The medium used for culturing the cells can also be selected appropriately based on the type of cells cultured and the intended use of the cultured cells.

The step of applying the external stimulus depends on the stimulus to which the shape-memory polymer responds. Various external stimuli include, but are not limited to, changes in temperature, pH, ionic strength, solvent, salt, surfactant, or an electric or magnetic field.

Temperature can be changed by placing the device in a temperature controlled enclosure. Temperature can also be changed by adding a solution of a desired temperature into the device.

pH and/or ionic strength can be changed by adding a solution of a desired pH or ionic strength into the device. pH and/or ionic strength can also be changed by adding certain compounds to the medium in which the cells are grown.

Electric and magnetic fields can be changed by placing the device in an appropriate electric or magnetic field.

In certain embodiments, the step of applying the stimulus is performed for a short period of time, for example, a few seconds to a few minutes. In one embodiment, the step of applying the stimulus is performed for about 5 seconds to about 10 minutes, about 15 seconds to about 8 minutes, about 30 seconds to about 5 minutes, about 1 minute to about 3 minutes, or about 2 minutes. In one embodiment, the step of applying the external stimulus is performed for about 5 seconds to about 30 seconds. As such, the step of applying the stimulus is performed rapidly thereby reducing the exposure of the cells to the changed conditions.

In another aspect, the present invention provides methods for in-situ quantification of cells on the shape-memory polymer surfaces. For example, the quantification may be accomplished utilizing a defined surface dimension, collecting the cells released from the surface and counting the cells obtained from the defined surface dimension.

The term “about” is used in this patent application to describe some quantitative aspects of the invention, for example, duration of applying a stimulus. It should be understood that absolute accuracy is not required with respect to those aspects for the invention to operate. When the term “about” is used to describe a quantitative aspect of the invention the relevant aspect may be varied by ±10% (for example, 1%, 2%, 3%, 4%, 5%, 6%, 7%, 8%, 9% or 10%).

### Materials and Methods

### Materials

NIH/3T3 mouse embryonic fibroblast cells were purchased from the American Type Culture Collection. Dulbecco's modified Eagle's medium (DMEM), Dulbecco's phosphate buffered saline (DPBS), newborn calf serum (NCS), 0.25% trypsin EDTA (1×), calcein AM, ethidium homodimer, penicillin and streptomycin were all purchased from Life Technologies. N-isopropylacrylamide (NIPAAm), 2-dimethoxy-2-phenylacetophenone (DMPA), N,N′-methylenebisacrylamide (MBAm), 3-(trichlorosilyl) propyl methacrylate (TPM), sodium azide (NaN₃), Rho-associated protein kinase (ROCK) inhibitor Y-27632, acetone and carbon tetrachloride were all purchased from Sigma-Aldrich. Methacryloxyethyl thiocarbonyl rhodamine B (Polyfluor™ 570) was purchased from Polysciences. 3,3′-dithiobissulfosuccinimidylpropionate (DTSSP) was obtained from Thermo Scientific. Silicone elastomer (PDMS) kits (Sylgard™ 184) were obtained from Dow Corning.

Preparation of Dynamic pNIPAAm Arrays

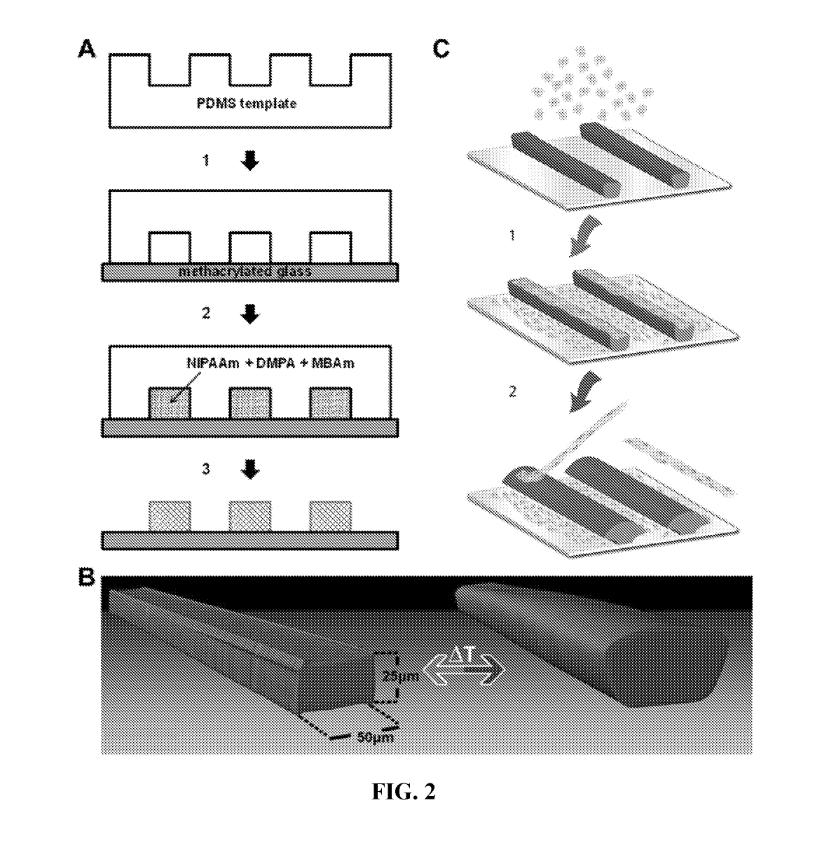

Patterns of crosslinked pNIPAAm (50-100 μm width×25 μm height×5 mm length) microbeams were fabricated on 22 mm×22 mm glass coverslips (#1.5) using PDMS molds by employing the micromolding in capillaries (MIMIC) technique (FIG. 2a). Briefly, the glass cover slip was surface modified with TPM in carbon tetrachloride. 1-4% MBAm crosslinker (5 mg/ml, 10% DMPA photo initiator (20 mg/ml) and 1% Polyfluor™ 570 (0.5 mg mg/ml) were added to a 250 mg/ml solution of NIPAAm in acetone. The resulting solution was introduced to the PDMS molds and polymerized with ultraviolet light (350 nm) for 4 min. The fabricated surfaces were sequentially rinsed with acetone, ethanol and water to remove unpolymerized monomer.

### Cell Culture

NIH/3T3 mouse embryonic fibroblast cells were cultured in 10% NCS growth medium containing 1% antibiotics (10,000 units/ml penicillin and 10,000 μg/ml streptomycin stock solution) at 37° C. in a humidified atmosphere of 5% CO₂. To prepare tissue modules, trypsinized fibroblasts were seeded onto the fabricated shape-memory polymer arrays at a density of 500-750 cells/mm² and cultured at 37° C. until confluence (24-48 h). Studies of release from low cell density (100 cells/mm²) were cultured for 24 h.

Release of Tissue Modules from Shape-Memory Polymers

Rapid release of tissue modules was induced by thermally initiated swelling of the shape-memory polymer beams. 2 ml of cold PBS (4-10° C.) was introduced (1 ml at a time) into the seeded dish containing 2.5 ml of 37° C. medium resulting in cooling to about 27° C. Cell release was monitored via time-lapse image acquisition on a microscope for at least 70 s.

### Viability of Cells Released

A cell viability assay was performed on released cells by using a LIVE/DEAD kit (containing calcein AM and ethidium homodimer) following the commercially recommended protocol. Once the cells reached confluence on the shape-memory polymer pattern, the tissue modules were released with fresh cold medium and plated onto a new tissue culture polystyrene (TCPS) dish. Following incubation for 24 or 48 h at 37° C., the cells were stained with 300 μl of 20 μM calcein AM and 40 μM ethidium homodimer-1 solution. After 30 min, the dish was rinsed twice with warm PBS and replenished with fresh medium prior to imaging.

### Mechanism of Tissue Module Detachment Studies

The mode of cell release from shape-memory polymer surfaces was examined by separately treating seeded samples with agents that modulate metabolism, contractility or adhesion. 24 h after attachment to microbeams, cells were exposed to sodium azide, a compound known to block ATP production via the inhibition of cytochrome C oxidase in mitochondria, Y-27632, a selective inhibitor of Rho-associated protein kinases, or DTSSP, a homobifunctional crosslinker that fixes only integrins bound to the extracellular matrix.

Briefly, samples were exposed to 2 mM sodium azide for 60 min or 50 mM Y-27632 or 2 mM DTSSP for 30 min prior to initiating tissue module release. To investigate the effect of surface strain on attached cells, the concentration of the network crosslinker (MBAm) in the prepolymer solution was varied from 1 to 4% before MIMIC processing. The one-dimensional width-wise strain in each microbeam was calculated from phase contrast micrographs as follows:

$$ɛ = \frac{Δ w}{{w}_{collapsed}} = \frac{{w}_{swollen} - {w}_{collapsed}}{{w}_{collapsed}} \qquad ( 1 )$$

where ε is the Cauchy strain, W_{collapsed} is the width of the polymer beam in the collapsed state and W_{swollen} is the width of the polymer beam in the swollen state. Cell detachment was calculated as the percent of cells released from the microbeams within 3 min after thermal stimulation.

### Microscopy

Video analysis (60 frames/second) and micrographs of samples were obtained using an Eclipse Ti-U (Nikon Instruments, Japan) fluorescent microscope equipped with a CCD camera (CoolSNAP HQ2, Photometrics, Tucson). Cell images were analyzed with NIS-Elements advanced research software Ver. 4.20 (Nikon Instruments) and cell counting was performed in ImageJ (NIH, USA). Images were processed to overlay fluorescent channels on the phase-contrast channel for LIVE/DEAD analysis.

To capture x-y-z image stacks for 3-D rendering of the microbeams, images were taken with a Leica TCS SP5 confocal laser scanning microscope (CLSM) equipped with 20×/0.7 NA and 40×/1.25 NA objectives (Leica Microsystems, Germany). An argon laser line, tuned to 543 nm, was applied to excite fluorescent microbeams and an acousto-optical beam splitter was used to filter the emission. Image sections were taken at a constant z-spacing of 0.25 μm and were captured with photomultiplier detectors using the Leica Application Suite Advanced Fluorescence software version 2.1.0 (Leica Microsystems, Germany).

### Statistical Analysis

Release data were collected with a sample size of n≥5 independent experiments for each case and reported as scatter plots. Statistical differences between treatment types were determined by performing a single factor analysis of variance followed by Tukey's test for pairwise comparisons with p<0.05 considered a significant difference.

Following are examples that illustrate procedures for practicing the invention. These examples should not be construed as limiting. All percentages are by weight and all solvent mixture proportions are by volume unless otherwise noted.

### Example 1—Surface-Confined Stimuli-Responsive Microbeams Swell Anisotropically

Shape-memory polymer microbeams were fabricated by MIMIC and photopolymerization (FIG. 2a). Rectangular prismatic microbeams with aspect ratios 0.25 to 0.5 were used to generate laterally straining surfaces in response to thermal activation. Under cell culture conditions at 37° C., the microbeams were collapsed polymer networks forming a stable topography suitable for culturing cells. Thermal reduction of the aqueous medium to 27° C. caused swelling of the surface-confined microbeams. Covalent attachment to the underlying substrate prevented expansion of the microbeams adjacent to the surface and resulted in anisotropic shape changes. The crosslink density in the polymer determined the extent of swelling; increasing the concentrations of the crosslinker reduced swelling and therefore retarded the lateral strain.

### Example 2—Rapid Tissue Module Release Via Surface Expansion of Shape-Memory Surfaces

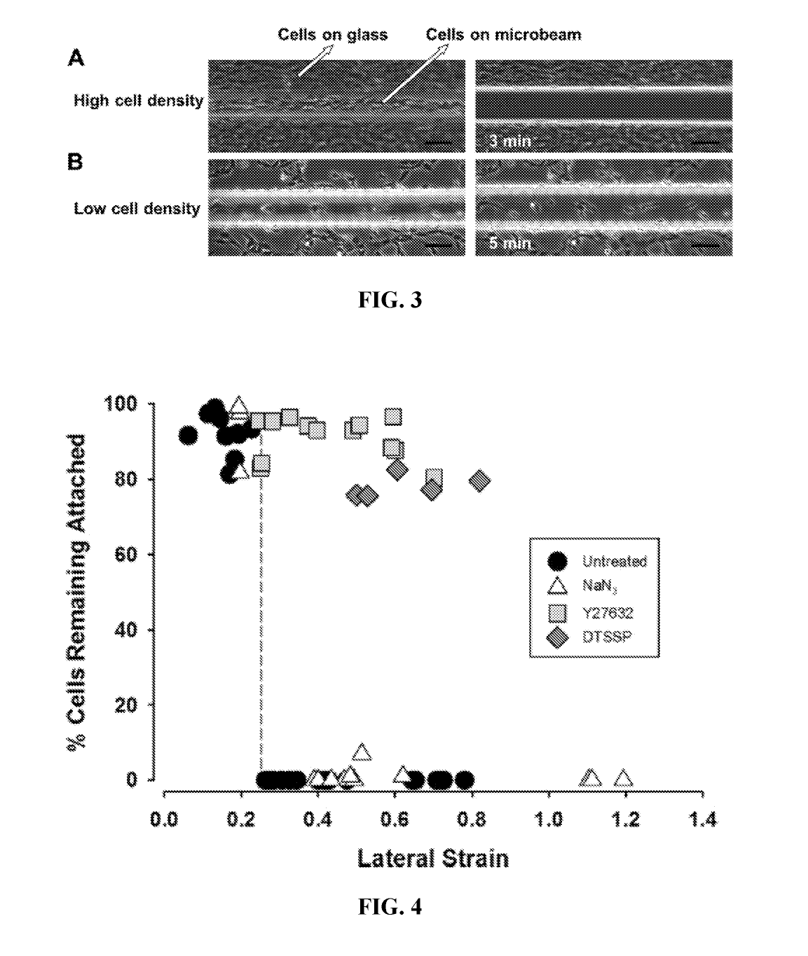

Tissue modules were formed from interconnected mouse embryonic fibroblasts by seeding these cells at high density (500 cell/mm²) onto arrays of shape-memory polymer microbeams crosslinked with 1% MBAm (n=5) (FIG. 2c). Seeded cells formed continuous multicellular monolayers that conformed to the microbeam's shape within 48 h. Cells were observed to populate the top surface of the microbeams and the recessed spaces between the beams (FIG. 3a).

The shape-memory property of these surface-confined microbeams was investigated as a method to harvest the attached tissue modules without disrupting their intercellular connections and organization. Thermal activation of these dynamic shape-memory polymer arrays triggered rapid release of tissue modules following swelling induced shape changes. Upon introduction of cold medium to reduce the temperature, the projected area of each microbeam increased as expansion occurred in the width-wise direction. Simultaneously, the confluent cells detached in aggregate from the microbeams as continuous tissue stripe modules (FIG. 3a). Cell-to-cell connections in the released tissue modules appeared to remain intact while cells between the microbeams maintained attachment to the glass surface after the change in temperature.

Release occurred rapidly as separation was observed within a few seconds of reducing the medium temperature to about 27° C. Complete detachment of the overlying tissue was observed in less than 3 min.

### Example 3—Effects of Cell Density on Tissue Module Release

Cell-to-cell connections were found to be a requirement for inducing confluent cell stripes to release from shape-memory polymer surfaces. For low cell density, 100 cells/mm² were seeded on microbeams (n=5) (FIG. 3b) and compared to high cell density samples (500 cells/mm²) with the same crosslink density (FIG. 3a). Cells seeded at low density were predominantly adhered individually to the microbeams with cells interacting with very few neighboring cells after 24 h of culture. Upon reducing the culture temperature, minimal detachment of cells was observed from low cell density microbeams (FIG. 3b). In contrast, complete detachment of continuous tissue modules occurred within seconds to minutes when the cells were connected atop the microbeams. A significant difference in the number of detached cells was observed between the high and low cell densities at 4 min after expansion (p<0.001).

The microbeams in these experiments were exposed to equal thermal stimuli and underwent similar lateral strains, indicating that the rapid release requires cell-to-cell connections and that the reduction in temperature alone does not induce tissue module release.

### Example 4—Mechanism of Tissue Release from Shape-Memory Polymer Microbeams

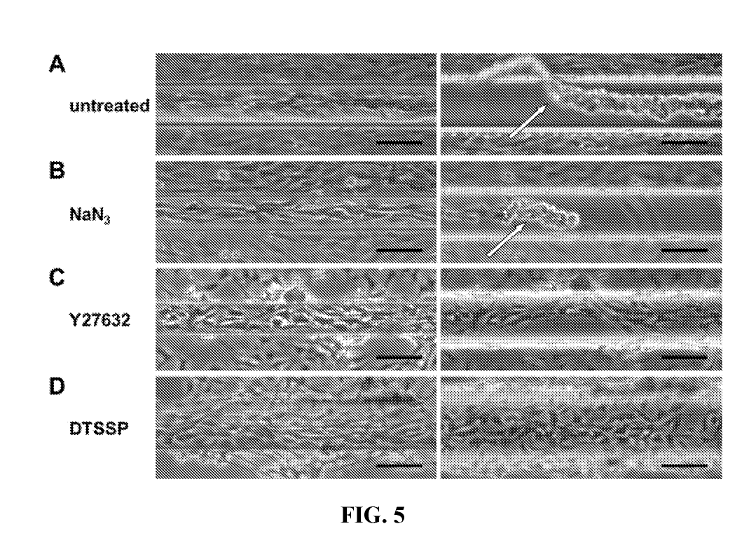

Based on the cell density results, it was hypothesized that a mismatch between the expanding surface and the allowable stress or strain in the tissue module caused the detachment of interconnected cells. To determine the mechanism of detachment from the shape-memory polymer microbeams, the mode of release was examined using four approaches. First, the degree of swelling of pNIPAAm shape-memory polymer is dependent on the extent of available network crosslinks; increasing crosslinks reduces swelling. The concentration of MBAm crosslinker in the prepolymer solution was varied from 1 to 4%, which resulted in microbeams whose surface expansion caused a range of lateral strain, ε, from 0.05 to 1.2 (i.e., 5-120% increase in width). Cells seeded at high density formed morphologically similar tissue modules on all crosslinked shape-memory polymer microbeams. Upon swelling, it was observed that lateral strain strongly regulated tissue release. Cells remained adhered and spread on microbeams with low strain; however, intact tissue modules were released above a threshold lateral strain of about 0.25 (p<0.001) (FIGS. 4 and 5a).

The roles of processes requiring cellular metabolism for cell detachment were examined by treating the tissue modules with sodium azide, a potent inhibitor of ATP production, prior to microbeam swelling. Less than 20% of the cells detached when ε<0.25 while more than 90% cell detachment was observed for ε>0.25 (FIGS. 4 and 5b). This trend was similar to untreated samples, indicating that inhibiting metabolic processes did not prevent cell release (p<0.001).

Since the mismatch between compliance of the tissue modules and the underlying surface expansion may disrupt the adhesive interface and lead to release, intracellular tension was modulated. Actin-myosin contractility was inhibited with Y-27632 prior to microbeam swelling. It was observed that the cells primarily remained attached, even after inducing large strains (ε>0.25), implying that contractility is required for rapid tissue module detachment (FIGS. 4 and 5c). Moreover, instead of releasing from the surface, the contractility-inhibited cells expanded with the swelling surface.

Attached cells were treated with DTSSP, a chemical crosslinker with a 12 Å spacer arm length which specifically crosslinks integrin receptors bound to extracellular matrix ligands. In this case, significant cell release was not observed within 5 min of initiating shape change, indicating that the disruption of integrin-mediated adhesion is required for release (FIGS. 4 and 5d).

Together, these results suggest that a mechanical mechanism triggers rapid tissue module release that is mediated by the lateral strain of the cell-surface interface provided by the shape changing properties of the patterned, surface-confined shape-memory polymer.

### Example 5—Strain-Induced Release does not Reduce Cell Viability

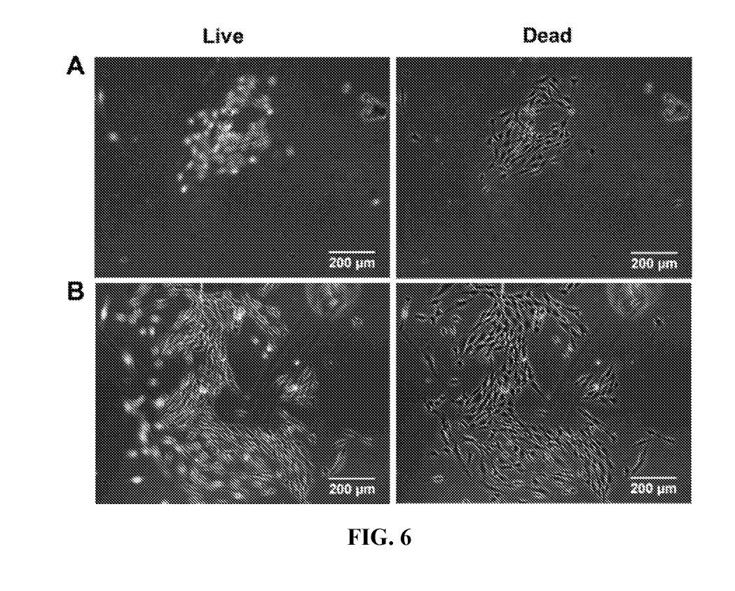

To examine the fate of cells within released tissue modules, the multicellular stripes were harvested via lateral strain (ε>0.30) and allowed to re-attach to tissue culture polystyrene (TCPS). Cell survival was observed over 48 h. After 24 h on TCPS, the organization of the tissue module was generally lost as the morphology became a loose aggregate of spreading cells, as expected for fibroblasts on TCPS (FIG. 6a). After 48 h, the cell number and area further increased (FIG. 6b). A LIVE/DEAD viability assay indicated that the majority (about 94%) of the harvested and reattached cells remained viable after release from the shape-memory polymer microbeams (FIG. 6).

### Example 6—the Devices of the Invention Provide Faster Release of Tissue Modules with Increased Cell Survival

Surface-confined shape-memory polymer structures were engineered that swell anisotropically when stimulated, for example, when the environment drops below the transition temperature. Micromolding was used to form these structures because it is amenable to parallel fabrication of patterned structures with diverse geometries and lateral dimensions spanning micrometers to centimeters on the same array, thereby greatly expanding the range of possible tissue module shapes and scales beyond what is currently possible. For this study, rectangular beams were used to create cell stripes, which facilitated the investigation of module release. Other tissue module geometries that have been fabricated include arbitrary 2-D shapes as well as thin lines and arcs.

Fibroblast-based tissue modules with defined geometries formed spontaneously when cells were seeded at high density atop the patterned arrays. Once formed, the harvest of these tissue modules to enable subsequent processing or modular assembly is provided. Release of tissue modules from pNIPAAm microbeams occurred within seconds and was completed within 3 min after lowering the culture temperature to 27° C. as long as there were cell-to-cell connections present and the lateral strains exceeded 25%. This 25% strain minimum was found to be a threshold for triggering release of fibroblast-based tissue modules.

A comparison to this surface strain effect is cyclic stretching of cells on elastic substrates. Studies using these experimental systems do not report instantaneous cell release; however, uniaxial stretch is typically less than 20%, designed to mimic physiological strains. Unlike enzymatic detachment techniques, the tissue modules released as single entities, while unconnected individual cells remained attached to the microbeams. Decreasing the density of cells attached to the shape-memory polymer microbeams significantly reduced cell release, even after inducing large strain (P50%). In contrast, cells seeded at high density to form confluent tissue modules detached rapidly and completely from microbeams subjected to similar lateral strains.

The release of the cells depended on the cohesive support from neighboring cells, suggesting that intercellular tension may play a significant role. This observation led to an investigation of the mechanism that regulates rapid tissue module release from these shape-changing surfaces. Importantly, the thermal stimulus used to actuate the pNIPAAm shape-memory polymer was not the key factor in achieving rapid cell release from the shape-memory polymer microbeams. When the amount of crosslinker was varied in the shape-memory polymer network, the extent of bulk swelling and, therefore, surface expansion was modulated. It was observed that lateral strain greater than 25% was required to initiate confluent tissue module detachment despite all modules being exposed to the same temperature reduction (about 10° C.).

Thus, the mechanism of rapid release from shape-memory pNIPAAm polymer beams appears to be dominated by mechanical expansion of the surface rather than a change in temperature or hydrophobicity.

When the mechanism of cell sheet lift-off was investigated, it was found that detachment from grafted pNIPAAm surfaces was reduced by about 50% in the presence of sodium azide, suggesting that metabolically driven processes are a key aspect to cell release from these surfaces. Additionally, both the depolymerization and stabilization of actin filaments reduced cell sheet detachment, indicating that actin dynamics also plays an important role. Based on these findings, it was proposed that cell detachment from grafted pNIPAAm surfaces due to a thermally induced shift in material properties was a two-step process: first, a passive detachment step, which resulted in the change in the surface interactions between the pNIPAAm layer and the cell-matrix construct; and second, a metabolically active detachment step, which requires cytoskeletal reorganization and intracellular signal transduction. Thus, an alternative release technique that requires only a small temperature shift, or other harmless stimulus, to trigger tissue module detachment in only seconds is desirable.

For the tissue modules formed on pNIPAAm microbeams, reducing metabolic activity by treatment with sodium azide had no detectable impact on tissue module detachment, in contrast to grafted pNIPAAm surfaces. Rather, the results suggest that the mechanism for detachment is strongly related to the degree of lateral strain from the anisotropic swelling of the polymer microbeams.

It is noted that no significant lateral strain occurs in thin grafted pNIPAAm films. Therefore, this strain dependent release may occur because the contractile forces within tissues and the stretching forces of the expanding surface are greater than the adhesion forces, and consequently mechanical failure occurred at the interface between the cells and the surface bound matrix, resulting in detachment.

Attached tissue modules were treated with Y-27632, an inhibitor of the p160ROCK Rho-associated protein kinase mediated actin-myosin contractility, or DTSSP, a homobifunctional crosslinker that only links integrin receptors that are bound to their extracellular ligand. Both prevented tissue module detachment for all strains tested (FIGS. 4 and 5) suggesting that the mechanical behavior of the tissue plays a significant role in detachment. Image analysis of cells on swollen microbeams after treatment with Y-27632 showed that cells deformed with the surface as the strain was applied, indicating greater compliance to stresses generated by the expansion of the underlying surface. Similarly, examining the DTSSP-treated samples showed cells with crosslinked adhesion receptors remained attached to the microbeams under large strains, indicating that release most likely occurs by breaking integrin-matrix adhesions. This is consistent with previous reports that indicate that at least some of the extracellular matrix is retained on pNIPAAm surfaces after cell release.

Thus, the release of patterned tissue modules from shape-memory polymer arrays occurs within seconds via a critical strain of the microbeams. Cell-to-cell junctions and cytoskeletal tension are required for complete detachment, and the separation occurs by disrupting adhesions between the fibroblasts and the extracellular matrix. Strain may disrupt the cell-matrix adhesive interface because inter- and intra-cellular tension prevents expansion of the tissue module with the swelling surface. Finally, the small (≤10° C.) and brief (≤3 min) thermal shift of the culture medium and the mechanical strain imposed on the tissue modules had minimal effect on cell integrity. Though some cells could have been lost during the transfer, a qualitative viability study following release showed that cells in large aggregates survive, adhere, spread and appeared to proliferate over 48 h, indicating that the rapid cooling and expansion of the support are not detrimental. Thus, the mechanically driven tissue module detachment from the shape-memory polymer allows for rapid release without enzymatic digestion or extended hypothermic incubation steps, thereby preserving cell health and cell-cell connections. Additionally, it enables the exploration of harvesting tissue building blocks for assembly into complex 3-D tissues.

### REFERENCES

[1] Elbert D L. Bottom-up tissue engineering. Curr Opin Biotechnol 2011; 22:674-80.
[2] Nichol J W, Khademhosseini A. Modular tissue engineering: engineering biological tissues from the bottom up. Soft Matter 2009; 5:1312-9.
[3] Muraoka M, Shimizu T, Itoga K, Takahashi H, Okano T. Control of the formation of vascular networks in 3D tissue engineered constructs. Biomaterials 2013; 34:696-703.
[4] Mcguigan A P, Sefton M V. Design and fabrication of sub-mm-sized modules containing encapsulated cells for modular tissue engineering. Tissue Eng 2007; 13:1069-78.
[5] Furth M E, Atala A, Van Dyke M E. Smart biomaterials design for tissue engineering and regenerative medicine. Biomaterials 2007; 28:5068-73.
[6] da Silva R M P, Mano J F, Reis R L. Smart thermoresponsive coatings and surfaces for tissue engineering: switching cell-material boundaries. Trends Biotechnol 2007; 25:577-83.
[7] Roy I, Gupta M N. Smart polymeric materials: emerging biochemical applications. Chem Biol 2003; 10:1161-71.
[8] Kumar A, Srivastava A, Galaev I Y, Mattiasson B. Smart polymers: physical forms and bioengineering applications. Prog Polym Sci 2007; 32:1205-37.
[9] Klouda L, Mikos A G. Thermoresponsive hydrogels in biomedical applications. Eur J Pharm Biopharm 2008; 68:34-45.
[10] Okano T, Yamada N, Sakai H, Sakurai Y. A novel recovery system for cultured cells using plasma-treated polystyrene dishes grafted with poly(Nisopropylacrylamide). J Biomed Mater Res 1993; 27:1243-51.
[11] Shibayama M, Tanaka T. Volume phase transition and related phenomena of polymer gels. In: Dušek K, editor. Responsive gels: volume transitions I. Berlin: Springer Verlag; 1993. p. 1-62.
[12] Canavan H E, Cheng X, Graham D J, Ratner B D, Castner D G. Cell sheet detachment affects the extracellular matrix: a surface science study comparing thermal liftoff, enzymatic, and mechanical methods. J Biomed Mater Res, Part A 2005; 75:1-13.
[13] Okano T, Yamada N, Okuhara M, Sakai H, Sakurai Y. Mechanism of cell detachment from temperature-modulated, hydrophilic-hydrophobic polymer surfaces. Biomaterials 1995; 16:297-303.
[14] Lee E L, von Recum H A. Cell culture platform with mechanical conditioning and nondamaging cellular detachment. J Biomed Mater Res, Part A 2010; 93:411-8.
[15] DuPont Jr S J, Cates R S, Stroot P G, Toomey R. Swelling-induced instabilities in microscale, surface-confined poly(N-isopropylacryamide) hydrogels. Soft Matter 2010; 6:3876-82.
[16] Hasegawa M, Yamato M, Kikuchi A, Okano T, Ishikawa I. Human periodontal ligament cell sheets can regenerate periodontal ligament tissue in an athymic rat model. Tissue Eng 2005; 11:469-78.
[17] Hayashida Y, Nishida K, Yamato M, Yang J, Sugiyama H, Watanabe K, et al. Transplantation of tissue-engineered epithelial cell sheets after excimer laser photoablation reduces postoperative corneal haze. Invest Ophthalmol Vis Sci 2006; 47:552-7.
[18] Yang G P, Soetikno R M. Treatment of oesophageal ulcerations using endoscopic transplantation of tissue-engineered autologous oral mucosal epithelial cell sheets in a canine model. Gut 2007; 56:313-4.
[19] Kolettis T M, Vilaeti A, Dimos K, Tsitou N, Agathopoulos S. Tissue engineering for post-myocardial infarction ventricular remodeling. Mini Rev Med Chem 2011; 11:8.
[20] Sekine H, Shimizu T, Hobo K, Sekiya S, Yang J, Yamato M, et al. Endothelial cell coculture within tissue-engineered cardiomyocyte sheets enhances neovascularization and improves cardiac function of ischemic hearts. Circulation 2008; 118:S145-52.
[21] St John Sutton M, Lee D, Rouleau J L, Goldman S, Plappert T, Braunwald E, et al. Left ventricular remodeling and ventricular arrhythmias after myocardial infarction. Circulation 2003; 107:2577-82.
[22] Dupont S J. Shape-shifting surfaces for rapid release and direct stamping of organized micro-tissues. Graduate school theses and dissertations; 2012 <http://scholarcommons.usf.edu/etd/4310>.
[23] Palmieri F, Klingenberg M. Inhibition of respiration under the control of azide uptake by mitochondria. Eur J Biochem 1967; 1:439-46.
[24] Ishizaki T, Uehata M, Tamechika I, Keel J, Nonomura K, Maekawa M, et al. Pharmacological properties of Y-27632, a specific inhibitor of rho-associated kinases. Mol Pharmacol 2000; 57:976-83.
[25] Dumbauld D W, Shin H, Gallant N D, Michael K E, Radhakrishna H, Garcia A J. Contractility modulates cell adhesion strengthening through focal adhesion kinase and assembly of vinculin-containing focal adhesions. J Cell Physiol 2010; 223:746-56.
[26] Gallant N D, Michael K E, Garcia A J. Cell adhesion strengthening: contributions of adhesive area, integrin binding, and focal adhesion assembly. Mol Biol Cell 2005; 16:4329-40.
[27] Wang J C, Thampatty B P. An introductory review of cell mechanobiology. Biomech Model Mechanobiol 2006; 5:1-16.
[28] Okano T, Yamada N, Sakai H, Sakurai Y. A novel recovery system for cultured cells using plasma-treated polystyrene dishes grafted with poly(Nisopropylacrylamide). J Biomed Mater Res 1993; 27:1243-51.
[29] Ohashi K, Yokoyama T, Yamato M, Kuge H, Kanehiro H, Tsutsumi M, et al. Engineering functional two- and three-dimensional liver systems in vivo using hepatic tissue sheets. Nat Med 2007; 13:880-5.
[30] Yang J, Yamato M, Shimizu T, Sekine H, Ohashi K, Kanzaki M, et al. Reconstruction of functional tissues with cell sheet engineering. Biomaterials 2007; 28:5033-43.
[31] Kwon O H, Kikuchi A, Yamato M, Sakurai Y, Okano T. Rapid cell sheet detachment from poly(N-isopropylacrylamide)-grafted porous cell culture membranes. J Biomed Mater Res 2000; 50:82-9.
[32] Masuda S, Shimizu T, Yamato M, Okano T. Cell sheet engineering for heart tissue repair. Adv Drug Deliv Rev 2008; 60:277-85.
[33] Shimizu T, Yamato M, Kikuchi A, Okano T. Two-dimensional manipulation of cardiac myocyte sheets utilizing temperature-responsive culture dishes augments the pulsatile amplitude. Tissue Eng 2001; 7:141-51.
[34] Shimizu T, Yamato M, Kikuchi A, Okano T. Cell sheet engineering for myocardial tissue reconstruction. Biomaterials 2003; 24:2309-16.
[35] Elloumi-Hannachi I, Yamato M, Okano T. Cell sheet engineering: a unique nanotechnology for scaffold-free tissue reconstruction with clinical applications in regenerative medicine. J Intern Med 2010; 267:54-70.
[36] Yamato M, Okano T. Cell sheet engineering. Mater Today 2004; 7:6.
[37] Fujita J. Cold shock response in mammalian cells. J Mol Microbiol Biotechnol 1999; 1:243-55.
[38] Sonna L A, Fujita J, Gaffin S L, Lilly C M. Invited review: effects of heat and cold stress on mammalian gene expression. J Appl Physiol 2002; 92:1725-42.
[39] Al-Fageeh M B, Marchant R J, Carden M J, Smales C M. The cold-shock response in cultured mammalian cells: harnessing the response for the improvement of recombinant protein production. Biotechnol Bioeng 2006; 93:829-35.
[40] M. Yamato, M. Okuhara, Fumiko Karikusa, Akihiko Kikuchi, Yasuhisa Sakurai, Teruo Okano. Signal transduction and cytoskeletal reorganization are required for cell detachment from cell culture surfaces grafted with a temperature responsive polymer. J Biomed Mater Res 1999; 44:8.
[41] Kim S J, Jun I, Kim D W, Lee Y B, Lee Y J, Lee J-H, et al. Rapid transfer of endothelial cell sheet using a thermosensitive hydrogel and its effect on therapeutic angiogenesis. Biomacromolecules 2013; 14:4309-19.
[42] Hyeong Kwon O, Kikuchi A, Yamato M, Okano T. Accelerated cell sheet recovery by co-grafting of PEG with PIPAAm onto porous cell culture membranes. Biomaterials 2003; 24:1223-32.
[43] Hou Y, Matthews A R, Smitherman A M, Bulick A S, Hahn M S, Hou H, et al. Thermoresponsive nanocomposite hydrogels with cell-releasing behavior. Biomaterials 2008; 29:3175-84.
[44] Haraguchi K, Takehisa T, Ebato M. Control of cell cultivation and cell sheet detachment on the surface of polymer/clay nanocomposite hydrogels. Biomacromolecules 2006; 7:3267-75.
[45] Reed J A, Lucero A E, Cooperstein M A, Canavan H E. The effects of cell culture parameters on cell release kinetics from thermoresponsive surfaces. J Appl Biomater Biomech 2008; 6:81-8.
[46] Gallant N D, Garcia A J. Quantitative analyses of cell adhesion strength. Methods Mol Biol 2007; 370:83-96 [Clifton, N.J.].
[47] Canavan H E, Cheng X, Graham D J, Ratner B D, Castner D G. Surface characterization of the extracellular matrix remaining after cell detachment from a thermoresponsive polymer. Langmuir 2005; 21:1949-55.
[48] Hu J, Meng H, Li G, and Ibekwe S. A review of stimuli-responsive polymers for smart textile applications. Smart Mater. Struct. 21 (2012) 053001.
[49] Meng H. A Brief Review of Stimulus-active Polymers Responsive to Thermal, Light, Magnetic, Electric, and Water/Solvent Stimuli. Journal of Intelligent Material Systems and Structures, vol. 21, no. 9859-885 (2010).

1. A device comprising a substrate and a pattern of shape-memory polymer fabricated upon the substrate, wherein the shape-memory polymer is converted to a deformed state by exposure to an external stimulus.

2. The device of claim 1, wherein the external stimulus is a change in temperature, pH, ionic strength, solvent, salt, surfactant, or an electric or magnetic field.

3. The device of claim 1, wherein the pattern of shape-memory polymer comprises a beam, disc, circle, ellipse, triangle, square, irregular shape, or repeated pattern thereof.

4. The device of claim 1, wherein the shape-memory polymer is poly(N-isopropylacrylamide).

5. The device of claim 1, wherein the substrate is produced from glass, silicone, polystyrene, polycarbonate or metal.

6. The device of claim 1, wherein the surface of the substrate and/or shape-memory polymer pattern is treated with an agent, wherein the agent facilitates the attachment of cultured cells onto the substrate and/or the shape-memory polymer pattern.

7. The device of claim 6, wherein the agent is fibronectin or collagen.

8. A method of releasing a patterned tissue module, the method comprising the steps of:
a) plating cells in a device comprising a substrate and a pattern of shape-memory polymer fabricated upon the substrate, wherein the shape-memory polymer is converted to a deformed state by exposure to an external stimulus,
b) culturing the cells under appropriate conditions to produce the patterned tissue module on the pattern of shape-memory polymer,
c) applying the external stimulus to the device, and
d) collecting the patterned tissue module released from the pattern of shape-memory polymer.

9. The method of claim 8, wherein the external stimulus is a change in temperature, pH, ionic strength, solvent, salt, surfactant, or an electric or magnetic field.

10. The method of claim 8, wherein the step of applying the stimulus is performed for about 5 seconds to about 10 minutes, about 15 seconds to about 8 minutes, about 30 seconds to about 5 minutes, or about 1 minute to about 3 minutes, or about 2 minutes.

11. The method of claim 8, wherein the step of applying the stimulus is performed for about 5 seconds to about 30 seconds.
